(19) United States
(12) Patent Application Publication
(10) Pub. No.: US 2021/0361513 A1
(43) Pub. Date: Nov. 25, 2021

(54) SPINAL TRACTION DEVICE

(71) Applicant: Jin Muck PARK, Gyeongsangnam-do (KR)

(72) Inventor: Jin Muck PARK, Gyeongsangnam-do (KR)

(21) Appl. No.: 16/963,331

(22) PCT Filed: Nov. 22, 2018
(86) PCT No.: PCT/KR2018/014464

§ 371 Date: Jul. 20, 2020

(30) Foreign Application Priority Data
Jan. 19, 2018 (KR) 10-2018-0007207
Aug. 8, 2018 (KR) 10-2018-0092590

(51) Int. Cl. A61H 1/02

(52) U.S. Cl. CPC A61H 1/0222; A61H 2203/0456; A61H 2230/855; A61H 2201/0192; A61H 2201/1238; A61H 2201/5066; A61H 2201/1604; A61H 2201/1628; A61H 2201/164; A61H 2201/1676; A61H 2201/1207

(57) ABSTRACT

A spinal traction device may include a base frame, a waist bed part on the upper side of the base frame to be movable up and down, an upper body bed part connected to one side of the waist bed part, and a lower body bed part connected to the other side of the waist bed part. The upper body bed part and the lower body bed part may be rotated with respect to the waist bed part as the waist bed part is raised and lowered. The lower body bed part may include a first lower body bed member and a second lower body bed member, the second lower body bed member is horizontally movable in the longitudinal direction of the lower body bed portion with respect to the first lower body bed member and is horizontally movable or rotatable in the width direction.

### TECHNICAL FIELD

The present invention relates to a spinal traction device. Specifically, the present invention relates to a spinal traction device, and more particularly, to a spinal traction device capable of effectively relieving fatigue of a musculoskeletal system such as back muscle, chest, and buttock as well as a spine while a user lies comfortably on a bed, and capable of aligning a human body correctly.

### BACKGROUND ART

In modern society, since a student studies while sitting on a chair for a long time due to competitions for an entrance examination, and computers and smartphones are increasingly used at workplace and home, the amount of time sitting on a chair during a day occupies a large proportion regardless of age or gender. Accordingly, the increase of patients having a scoliosis, a lumbar disc disease, a turtleneck, or the like, which is changed in a spinal shape, are becoming a social issue. In order to solve the above problems, Korean Registered Patent Publication No. 10-1329233 has been registered. However, since a user who uses the spinal traction device cannot feel comfortable while using the spinal traction device, the usability of the spinal traction device may be lowered, and it is difficult to adjust the degree of spinal traction, a length or the like suitable for the user. In addition, since the spine stretching exercise device according to the registered patent publication has a complicated structure, it is uneconomical due to the increase in manufacturing costs. In addition, a bed having a curved part is uncomfortable when the user lies thereon, a roller moving while rolling on a support of a base frame fails to solve a noise problem, and foreign substances stuck in exposed rollers and supports may cause an abnormal operation.

### DISCLOSURE

### Technical Problem

The present invention is proposed to solve all the problems as described above. An object of the present invention is to provide a spinal traction device safely used in a more comfortable posture, having a simple structure to reduce manufacturing costs, being sturdy without breakage and breakage, and remarkably increasing a correction effect.

In addition, the embodiments of the present invention provide a spinal traction device capable of increasing a correction effect on a lower body of a user by diversifying the degree of driving freedom of a lower bed part.

In addition, the embodiments of the present invention provide a spinal traction device for controlling the degree of ascending and descending of a waist bed part so as to be suitable for a body condition of the user through a sensor unit provided in the lower bed part to provide an optimal spinal correction effect according to the user and minimize the risk of injury to the user.

In addition, the embodiments of the present invention provide a spinal traction device capable of providing a customized spinal correction effect according to the user by measuring the degree or scoliosis or lordosis of the user's spine through a spine scan unit.

In addition, the embodiments of the present invention provide a spinal traction device capable of independently rotating only an upper bed part when needed by the user to diversify a spinal traction scheme.

### Technical Solution

One embodiment of the present invention provides a spinal traction device including: both supports having guide rails in front and rear directions of an inner surface thereof; a lower bracket mounted therein with a cylinder and a guide rod that are fixed between the both supports and vertically operated; an upper bracket for fixing the cylinder and the guide rod onto the lower bracket; an upper bed and a lower bed provided with hinges in front and rear directions of the upper bracket and rotated about the hinges, respectively; and connecting rods having rollers attached to lower ends on both sides of middle parts of the upper bed and the lower bed in which the rollers move along the guide rails, respectively.

Another aspect provides a spinal traction device, in which a spine support may be provided between the upper bed and the lower bed and cushions may be provided on both sides of the spine support.

Another aspect provides a spinal traction device, in which the upper bed may be formed therein with a space part, and provided with a headrest moving along guide grooves on both sides of the space part.

In still another aspect, one embodiment of the present invention provides a spinal traction device including: a base frame; a waist bed part provided on an upper side of the base frame so as to be movable up and down with respect to a ground to support a waist of a user; an upper bed part connected to one side of the waist bed part to support an upper body of the user; and a lower bed part connected to an opposite side of the waist bed part to support a lower body of the user, wherein the upper bed part and the lower bed part are rotated at a predetermined angle with respect to the waist bed part when the waist bed part ascends and descends, the lower bed part includes a first lower bed member for supporting a thigh of the user, and a second lower bed member for supporting a calf portion of the user, and the second lower bed member is horizontally movable in a longitudinal direction of the lower bed part with respect to the first lower bed member and horizontally movable or horizontally rotatable in a widthwise direction perpendicular to the longitudinal direction.

In another aspect, the second lower bed member may include: a second lower bed body; and a length adjusting part for connecting the first lower bed member to the second lower bed body, wherein the length adjusting part may be horizontally movable in the longitudinal direction of the lower bed part from one side of the first lower bed member.

In another aspect, the first lower bed member includes: a first lower bed body; and a rotation guide part formed on one side of the first lower bed body to guide a horizontal rotation of the second lower bed member, wherein the length adjusting part may be rotatably fastened to one end of the first lower bed body and at least a part of the rotation guide part along the rotation guide part so as to be horizontally rotatable in the widthwise direction of the lower bed part.

In another aspect, the second lower bed body may include: a pair of calf support members spaced apart from each other to support calves of the user, respectively; and a horizontal slide part capable of horizontally moving the pair of calf support members in the widthwise direction of the lower bed part from one side of the length adjusting part.

In another aspect, the second lower bed body may further include sensor units positioned on the pair of calf support members to detect distances between the calf support members and the calves of the user, respectively.

In another aspect, an upper surface of each of the pair of calf support members may be concave downward to surround the calf of the user.

In another aspect, horizontal positions of the pair of calf support members may be higher than a horizontal position of the first lower bed member with respect to the ground.

In another aspect, the upper bed part may include: a first upper bed member for supporting a back of the user; a second upper bed member connected to the first upper bed member to support an occipital part of the user; and a driving unit for rotating the first upper bed member by a predetermined angle with respect to the waist bed part.

In another aspect, the upper bed part may further include a spine scan unit provided to be movable along the spine of the user on the first upper bed member to detect a degree of scoliosis or lordosis of the spine.

In another aspect, the spine scan unit may include: a guide groove; and a scan member movable along the spine of the user through the guide groove.

In another aspect, the scan member may be three-dimensionally movable in x-axis and y-axis directions orthogonal to each other on a plane of the first upper bed member and a z-axis direction orthogonal to the plane of the first upper bed member.

In another aspect, the waist bed part may include: a waist bed body; and a pelvic support provided on the waist bed body so as to be moved toward a pelvis of the user when the waist bed part ascends.

### Advantageous Effects

According to the embodiments of the present invention, guide rails and rollers for operating a bed can be prevented from being exposed to prevent foreign substances from being stuck so as to be safe without easily having a trouble, the weight distribution due to a central connecting rods can cause a smooth operation, the durability can be remarkably increased due to a sturdy structure, and the spine can be corrected while a body fixing strength can be increased and a comfortable posture can be maintained through a waist fixing device and a headrest.

In addition, according to the embodiments of the present invention, the lower body correction effect onto the user can be increased by diversifying the degree of driving freedom of a lower bed part.

In addition, according to the embodiments of the present invention, the degree of ascending and descending of a waist bed part is controlled to be suitable for a body condition of the user through a sensor unit provided in the lower bed part, so that an optimal spinal correction effect can be provided according to the user, and the injury risk onto the user can be minimized.

In addition, according to the embodiments of the present invention, the degree or scoliosis or lordosis of the user's spine may be measured through a spine scan unit, so that a customized spinal correction effect can be suitably provided to the user.

In addition, according to the embodiments of the present invention, only the upper bed part may be independently rotated and operated according to the user's needs, so that the spinal traction scheme can be diversified.

### BRIEF DESCRIPTION OF THE DRAWINGS

FIG. 1 is a perspective view of a spinal traction device according to one embodiment of the present invention.

FIG. 2 is a front view of the spinal traction device according to one embodiment of the present invention.

FIG. 3 is a front view showing an internal structure of the spinal traction device according to one embodiment of the present invention.

FIG. 4 is a view showing a state of use of the spinal traction device according to one embodiment of the present invention.

FIG. 5 is an enlarged sectional view of the internal structure of the spinal traction device according to one embodiment of the present invention.

FIG. 6 is a central sectional view of the spinal traction device according to one embodiment of the present invention.

FIG. 7 is a sectional view taken along line A-A in FIG. 2.

FIG. 8 is a schematic view showing the spinal traction device according to another embodiment of the present invention when viewed from a side thereof.

FIGS. 9a and 9b are views showing that an upper bed part and a lower bed part are rotated as a waist bed part of the spinal traction device according to another embodiment of the present invention moves up and down.

FIG. 10 is a view schematically showing the degree of driving freedom of the lower bed part of the spinal traction device according to another embodiment of the present invention.

FIG. 11a is a view schematically showing a first driving of the lower bed part of the spinal traction device according to another embodiment of the present invention when viewed from the side thereof.

FIG. 11b is a view schematically showing a second driving of the lower bed part of the spinal traction device according to another embodiment of the present invention when viewed from the top.

FIG. 11c is a view schematically showing a third driving of the lower bed part of the spinal traction device according to another embodiment of the present invention when viewed from the front (y-axis direction in FIG. 10).

FIG. 12 is a view showing a driving of the upper bed part of the spinal traction device according to another embodiment of the present invention.

FIG. 13 is a perspective view showing the upper bed part of the spinal traction device according to another embodiment of the present invention.

FIGS. 14(a) to 14(c) are photographs showing examples of a used state of the spinal traction device according to another embodiment of the present invention.

FIG. 15(a) is an x-ray photograph showing a state of a spine before using the spinal traction device according to another embodiment of the present invention.

FIG. 15(b) is an x-ray photograph showing a state of the spine according to another embodiment of the present invention when a user uses the spinal traction device in a supine posture.

FIG. 15(c) is an x-ray photograph showing a state of the spine when the user uses the spinal traction device in a prone posture according to another embodiment of the present invention.

### BEST MODE

Hereinafter, specific embodiments of the present invention will be described with reference to the drawings. However, the descriptions are merely the exemplary illustrations and the present invention is not limited thereto.

For describing the present invention, the detailed description of the known art related to the present invention incorporated herein will be omitted when it possibly makes the subject matter of the present invention unclear unnecessarily. In addition, the terms described later are terms defined in consideration of functions in the present invention, which may vary according to a user or operator's intention or practice. Therefore, the definition shall be made based on the contents throughout the specification herein. The technical idea of the present invention shall be determined by the following claims. The following embodiments are merely means for efficiently describing the technical idea of the present invention to those skilled in the art of the present invention.

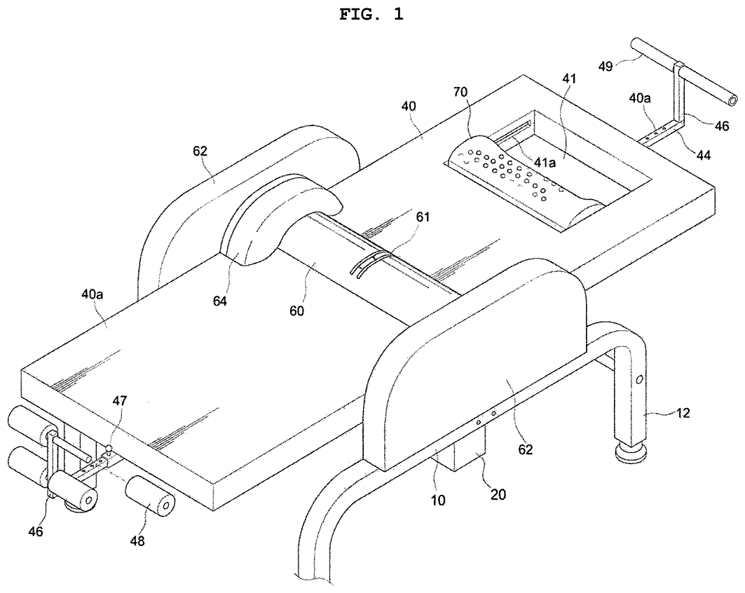

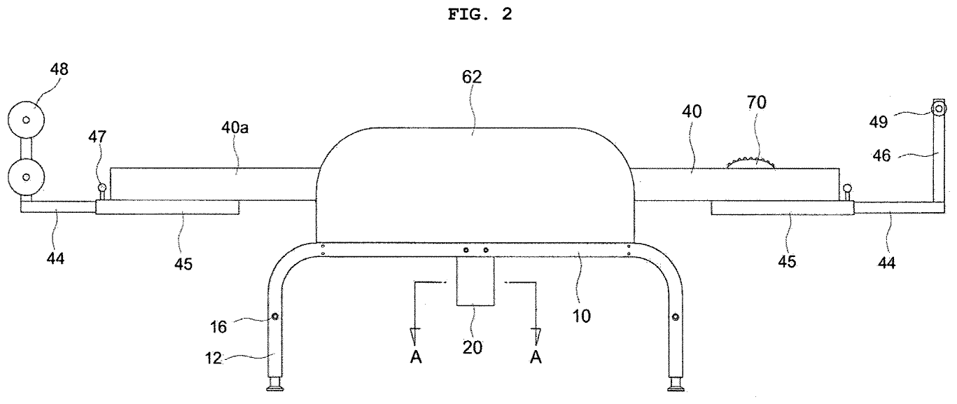

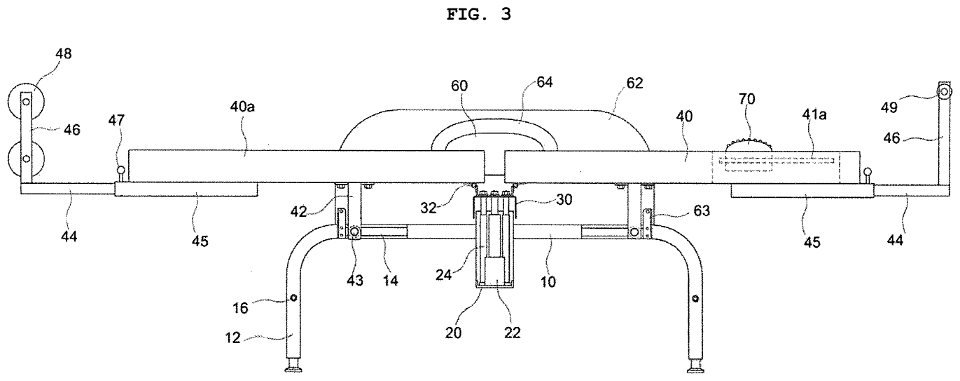

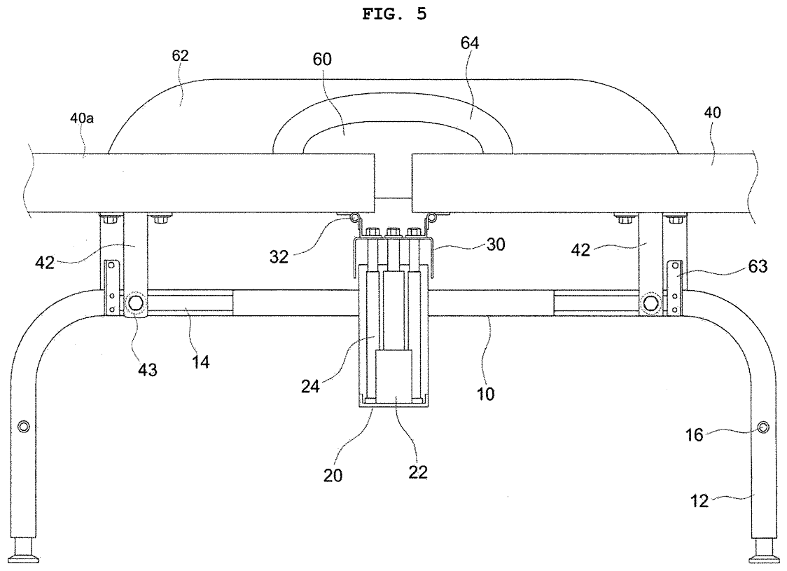

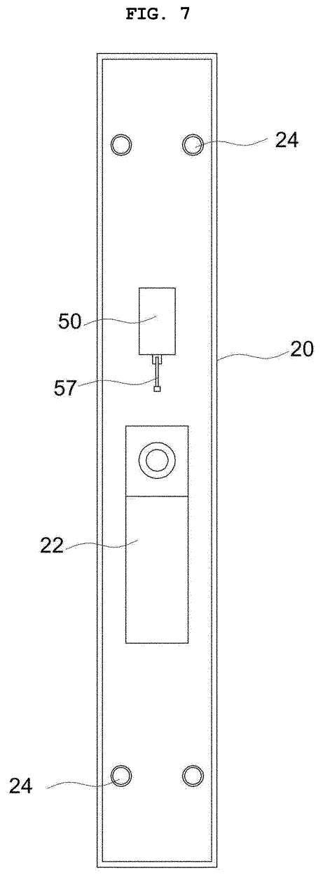

FIGS. 1 to 7 show a spinal traction device according to the embodiments of the present invention. The spinal traction device according to the drawings includes: supports 10 provided on both sides and having guide rails 14 in front and rear directions of an inner surface; a lower bracket 20 fixed between the supports on the both sides and equipped with a cylinder and a guide rod that operate vertically; an upper bracket 30 for fixing the cylinder and the guide rod on the lower bracket 20; an upper bed 40 and a lower bed 40a provided with hinges 32 in front and rear directions of the upper bracket and rotated about the hinges, respectively; and connecting rods 42 having rollers attached to lower ends on both sides of middle parts of the upper bed 40 and the lower bed 40a in which the rollers move along the guide rails, respectively.

In addition, a spine support is provided between the upper bed and the lower bed and cushions are provided on both sides thereof, in which a space part 41 is formed in the upper bed, and provided with a headrest 70 moving along guide grooves on both sides of the space part.

The above description will be described in more detail as follows.

The spinal traction device according to the present invention has a structure that the upper bed 40 is separated from the lower bed 40a about the side plates 62 on both sides, a center is moved upward by the vertical movement of the cylinder thereunder, and an upper end and a lower end are rotated downward.

First, supports 10, which are installed at both sides parallel to each other and each have legs 12 formed in front/rear directions, are provided, in which guide rails 14 are formed at a front and a rear of inner sides of the supports. In addition, a lower bracket 20 having a predetermined space therein is fixed between the both supports 10, and upper bracket 30 is provided thereon. A cylinder 22 vertically operated is installed between the lower bracket 20 and the upper bracket 30 to move the upper bracket 30 up and down according to the operation of the cylinder. In addition, at least one guide rod 24 is provided between the lower bracket 20 and the upper bracket 30 to facilitate the vertical operation of the upper bracket together with the cylinder.

In addition, the upper bracket 30 for fixing the cylinder and the guide rod onto the lower bracket 20 is equipped with hinges 32 in front and rear directions, and the upper bed 40 and the lower bed 40a rotated about the hinges are assembled thereto. Accordingly, a rear of the upper bed 40 is coupled to the hinge to allow a front to be rotated up and down, and a front of the lower bed 40a is coupled to the hinge to allow a rear to be rotated up and down.

Connecting rods 40 are fixed to both sides of middle parts of the upper bed 40 and the lower bed 40a to support the bed 40 and 40a, respectively, in which rollers 43 are attached to lower ends of the connecting rods 42 and placed on the aforementioned guide rails 14 so as to be rotated and moved. Accordingly, when each bed is moved vertically by the cylinder the bed is smoothly rotated while the other side is horizontally moved. In addition, a spine support 60 is provided between the upper bed 40 and the lower bed 40a, and cushions are provided on both sides to comfortably support a waist, in which each bed may be provided therein with a heating device and an acupressure device. The spine support 60 is formed at a center thereof with a spine guide groove 61 to maintain the spine so as to prevent the spine from twisting in the middle when a user lies thereon. In addition, the cushions 64 are provided on both sides of the spine support 60 to fix a posture in close contact with a waist part and enable the user to feel safer and more comfortable.

The cushion 64 may be formed of an elastic material such as rubber or sponge, and may have a structure provided with an airbag to or from which air is injected or removed to fit a human body.

Next, a space part 41 is formed in the upper bed, and provided therein with a headrest 70 moved according to a height of the user. The headrest 70 may be preferably formed of an elastic material, may include acupressure protrusions, and provided on both sides thereof with pins 72, in which guide grooves 41a, to which the pins are inserted and moved, are formed on both side surfaces of the space part 41.

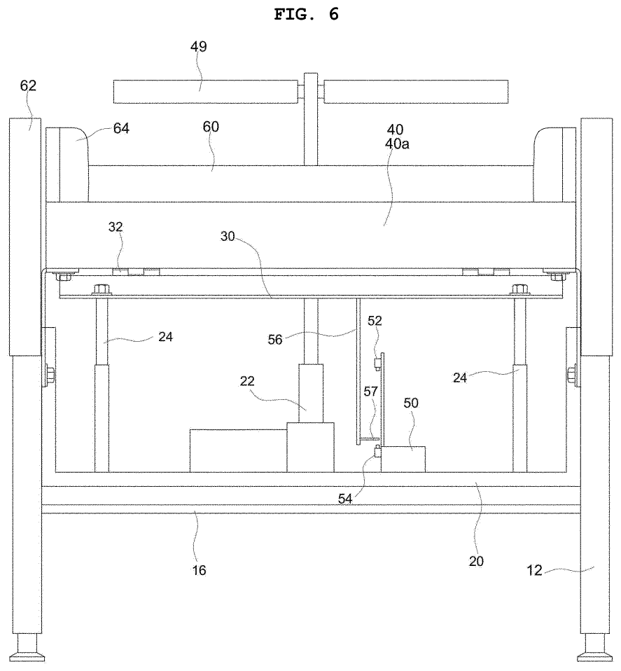

In addition, the upper bed 40 is provided at a front center thereof with a handle 49, and the lower bed 40a is provided with foot fixing rollers 48. The handle 49 and the foot fixing rollers 48 are fixed to centers of the beds through fixing tube members 45, respectively, and assembled by horizontal rods 44 pushed in and pulled out to adjust a length according to the height of the user. In addition, a stopper 47 capable of fixing a position of the horizontal rod is provided. In addition, a limit switch 50 is provided above the lower bracket 20 as shown in FIG. 6. The limit switch 50 is configured to limit a moving distance of each bed, has an upper contact part 52 and a lower contact part 54, and is controlled by an operation pin 57 attached to the connecting rod 56 fixed to the upper bracket 30 that are vertically moved.

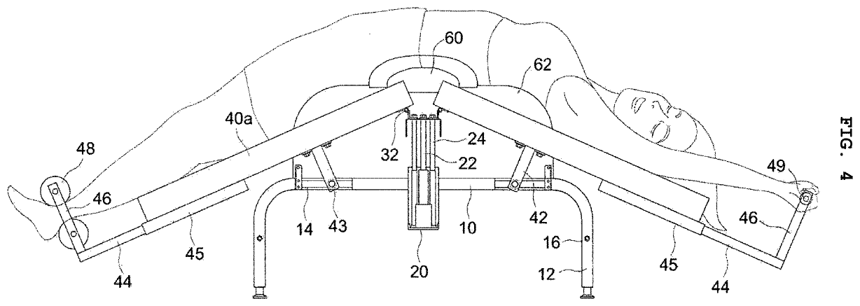

The spinal traction device having the above configuration according to the embodiments of the present invention are operated as follows. First, after the upper bed 40 and the lower bed 40a are set to be a horizontal state, the user lies thereon and fits and fixes feet to foot fixing rollers 48. Then, when the cylinder 22 is raised upward by manipulating an operation switch (not shown in the drawing) provided on the lower bracket 20 or the side plate 62, ends of inner sides of the beds are raised upward about the connecting rod 42, and the other sides are rotated downward, so that the spine of the user is retracted while the waist is bent upward as shown in FIG. 4, thereby implementing effects of correction and treatment. Then, when the cylinder descends, one operation cycle is completed, and the user may use the spinal traction device suitably for the user while adjusting the number of times and the delay time according to control settings.

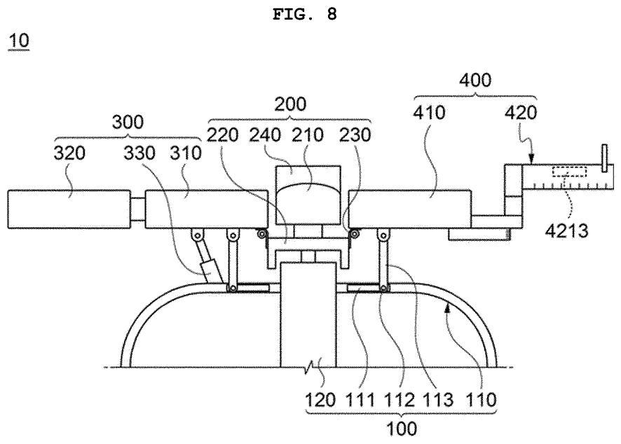

FIG. 8 is a schematic view showing the spinal traction device 10 according to an embodiment of the present invention when viewed from a side thereof.

Referring to FIG. 8, the spinal traction device 10 according to one embodiment of the present invention may include: a base frame 100; a waist bed part 200 that supports a waist of a user; an upper bed part 300 that supports an upper body of the user; and a lower bed part 400 that supports a lower body of the user Specifically, the above-described upper bed part 300 may include a first upper bed member 310 positioned adjacent to the waist bed part 200 to support a back part of the user, and a second upper bed member 320 connected to the first upper bed member 310 to support an occipital part of the user. The above-described lower bed part 400 may include a first lower bed member 410 positioned adjacent to the waist bed part 200 to support a thigh portion of the user, and a second lower bed member 420 that supports a calf portion of the user.

The above-described waist bed part 200 may be configured to move up and down over an upper side of the base frame with respect to the ground, and the upper bed part 300 and the lower bed part 400 may be rotated at a predetermined angle relative to the waist bed part 200 when the waist bed part 200 moves up and down

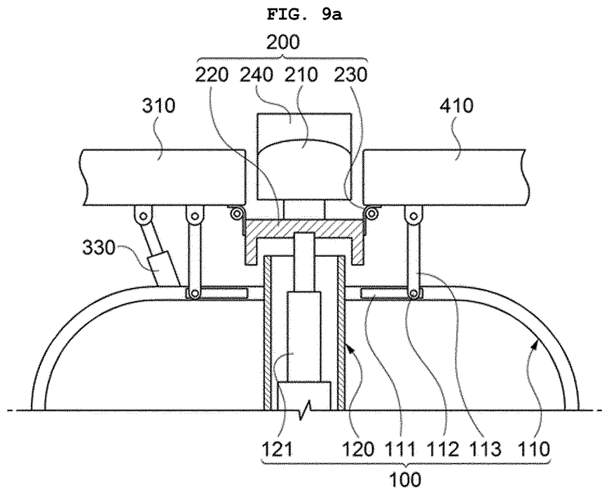

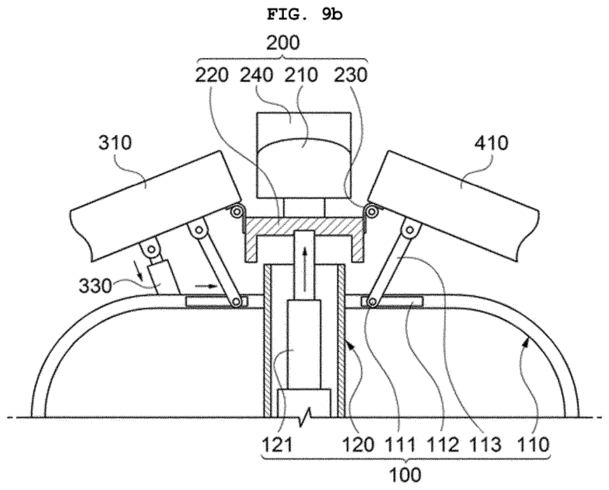

FIGS. 9a and 9b are views showing that the upper bed part 300 and the lower bed part 400 are rotated as the waist bed part 200 of the spinal traction device 10 moves up and down according to one embodiment of the present invention.

Referring to FIGS. 9a and 9b, the above-described base frame 100 may include a support frame 110 seated on the ground to support a structure of the spinal traction device 10, a plurality of link members 113 having ends connected to the first upper bed member 310 and the first lower bed member 410 and the other ends connected to the support frames 110, respectively, rollers 112 coupled to the other ends of the link members 113, and rail grooves 111 formed on the support frame 110 to guide the above-described rollers 112.

One end of the link member 113 may be hinged to one side of the first upper bed member 310 or the second lower bed member 420. In addition, the other end of the link member 113 may be positioned to be horizontally movable along the rail groove 111 on the support frame 110 through the roller 112.

In addition, the base frame 100 may further include an ascending and descending driving unit 120 positioned below the waist bed part 200, and the ascending and descending driving unit 120 may be provided therein with a cylinder 121. The above-described cylinder 121 may extend in a direction perpendicular to the ground, and may be coupled to the waist bed part 200 at the top, so that the waist bed part 200 may be moved up or down according to the driving of the cylinder 121.

The above-described cylinder 121 may be formed of a screw jack having a screw (screw shaft)-type cylinder 121 reciprocating by rotation of an electric motor, a hydraulic or pneumatic cylinder 121, or the like, but this is merely illustrative and the present invention is not limited thereto.

In addition, the above-described waist bed part 200 may include a waist bed member 210 that supports the waist of the user, an ascending and descending member 220 disposed below the waist bed member 210 and coupled to the upper end of the above-described cylinder 121 so as to be moved up and down in a direction perpendicular to the ground when the cylinder 121 is driven, and a hinge member 230 provided on the ascending and descending member 220 and hinged to the first upper bed member 310 and the first lower bed member 410.

The above-described hinge member 230 may include a plurality of hinge members so as to be hinged to each of the first upper bed member 310 and the first lower bed member 410 at both sides in the horizontal direction of the ascending and descending member 220. In addition, an upper surface of the above-described waist bed member 210 may be formed in a curved shape to be convex upward so that the user feels comfortable.

Specifically, the rotational driving of the upper bed part 300 and the lower bed part 400 according to the ascending and descending of the above-described lower bed part 400 will be described. As shown in FIG. 9a, at first, the first lower bed part 400 and the second lower bed part 400 may be positioned parallel to the ground. Then, the cylinder 121 of the above-described ascending and descending driving unit 120 may be operated according to the operation of the spinal traction device 10 according to one embodiment of the present invention. When the ascending and descending member 220 is elevated by driving the cylinder 121, the rollers 112 fastened to the other ends of the link members 113 may be moved along the rail grooves 111 toward the ascending and descending driving unit 120 at the center thereof, and each of the first upper bed member 310 and the first lower bed member 410 hinged to one ends of the link members 113 may be rotated and lowered by a predetermined angle around hinge shafts of the above-described hinge members 230, respectively.

In other words, the waist bed member 210 may also ascend when the ascending and descending member 220 ascends, and the first upper bed member 310 and the second lower bed member 420 may be rotated and lowered by a predetermined angle relative to the waist bed part 200 through the hinge members 230 while one sides are constrained to the base frame 100, respectively. Although the second upper bed member 320 and the second lower bed member 420 are not shown in the drawing, it is obvious to those skilled in the art that the second upper bed member 320 and the second lower bed member 420 connected to the first upper bed member 310 and the first lower bed member 410 may also be rotated together, respectively, when the first upper bed member 310 and the first lower bed member 410 are rotated downward.

In addition, the above-described hinge member 230 may have a predetermined elasticity in a radial direction around the hinge shaft, and the first upper bed member 310 and the first lower bed member 410 may be easily rotated upward when the ascending and descending member 220 descends by driving the cylinder 121.

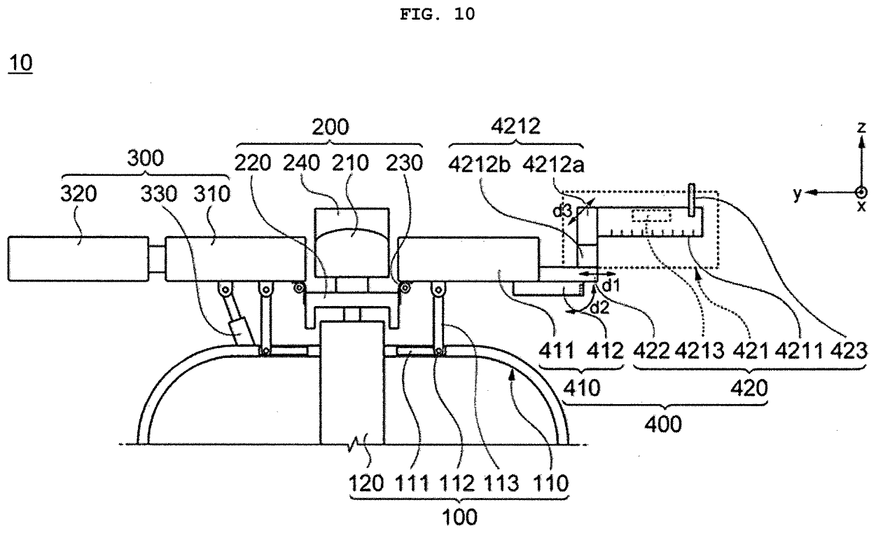

FIG. 10 is a view schematically showing the degree of driving freedom of the lower bed part 400 of the spinal traction device 10 according to an embodiment of the present invention.

Referring to FIG. 10, as described above, the lower bed part 400 of the spinal traction device 10 according to one embodiment of the present invention may include a first lower bed member 410 positioned adjacent to the waist bed part 200 to support a thigh portion of the user, and a second lower bed member 420 connected to the first lower bed member 410 to support a calf portion of the user. The above-described second lower bed member 420 may be horizontally movable in a longitudinal direction (y-axis direction in the drawing) of the lower bed part 400 with respect to the first lower bed member 410 (d1—a first driving of the lower bed part 400), horizontally rotatable in a widthwise direction (x-axis direction in the drawing) perpendicular to the longitudinal direction (d2—a second driving of the lower bed part 400), or horizontally movable in the widthwise direction (x-axis direction) (d3—a third driving of the lower bed part 400).

The present invention is not limited to only one driving scheme among the first driving d1, the second driving d2 and the third driving d3 of the above-described lower bed part 400, and one or more driving schemes may be applicable. Preferably, all of the first driving d1, the second driving d2 and the third driving d3 of the lower bed part 400 may be provided. Each driving scheme will be described later in detail.

Specifically, the detailed configuration of the lower bed part 400 will be described as follows. The above-described second lower bed member 420 may include a second lower bed body positioned adjacent to the first lower bed member 410 to support the calf portion of the user and a length adjusting part 422 for connecting the first lower bed member 410 to the second lower bed body.

The second lower bed body may include a pair of calf support members 4211 spaced apart from each other to support calves of the user, respectively, and a horizontal slide part 4212 capable of horizontally moving the pair of calf support members 4211 in the widthwise direction of the lower bed part 400 from one side of the length adjusting part 422. The above-described pair of calf support members 4211 may have one end coupled to at least a part of the horizontal slide part 4212, and the horizontal slide part 4212 may be coupled to one side of the length adjusting part 422.

In addition, the above-described first lower bed member 410 may include a first lower bed body 411 for supporting a thigh portion of the user, and a rotation guide part 412 formed on one side of the first lower bed body 411 to guide the horizontal rotation of the second lower bed member 420.

Further, the second lower bed body of the spinal traction device 10 according to one embodiment of the present invention may further include sensor units 4213 positioned on the pair of calf support members 4211 to detect distances between the calf support members 4211 and the calves of the user, respectively, and ankle fixing parts 423 positioned at one ends of the calf support members 4211 to fix and support ankle parts of the user toward the calf support members 4211, respectively.

Specifically, when the user uses the spinal traction device according to one embodiment of the present invention, an upper body and a lower body of the user descend as the waist bed part 200 ascends. The calf portion in the user's lower body may be spaced apart from the calf support member 4211 by a predetermined distance while an ankle part is fixed to the ankle fixing part 423 due to a body structure.

When the waist bed part 200 excessively ascends to burden the user's body, an unexpected injury such as spinal disc damage may occur. The spinal traction device 10 according to one embodiment of the present invention may detect the degree of calf separation of the user through the above-described sensor unit, so that the ascending operation of the waist bed part 200 may be stopped when the user's calf is spaced by a predetermined interval or more.

In other words, the ascending and descending of the waist bed part 200 may be controlled through the sensor unit, so that a customized spine correction effect can be provided according to the user, and unexpected injuries and the like during using the spinal traction device 10 can be prevented. In addition, the above-described calf support member 4211 is provided in a pair corresponding to the calves of the user, respectively, and the above-described sensor units 4213 are provided on the pair of calf support members 4211, respectively, so that the degree of separation of the both calves of the user from the calf support members 4211 can be measured, respectively, and the degree of postural twist of the user can be checked based on the measured result.

In addition, horizontal positions of the pair of calf support members 4211 may be higher than a horizontal position of the first lower bed member 410 with respect to the ground.

Specifically, when the first lower bed member 410 and the second lower bed member 420 are positioned parallel to the ground, a height of the calf support member 4211 of the second lower bed member 420 may be higher than a height of the first lower bed member 410. In general, when a person lies parallel to the ground, the calf portion is naturally separated from the ground due to the human body structure. The pair of calf support members 4211 are formed higher with respect to the ground on the second lower bed member 420, so that a comfort may be provided to the user during using the spinal traction device 10 according to one embodiment of the present invention.

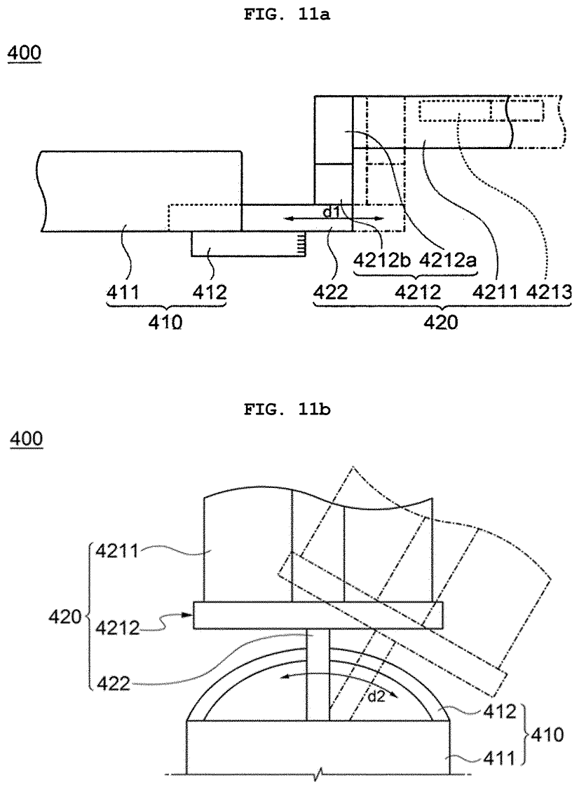

FIG. 11a is a view schematically showing a first driving of the lower bed part 400 of the spinal traction device 10 according to one embodiment of the present invention when viewed from the side thereof.

Referring to FIG. 11a, the above-described length adjusting part 422 may be configured to be horizontally movable in the longitudinal direction (y-axis direction) of the lower body bed part 400 from one side of the first lower bed body 411.

Specifically, the horizontal moving part may be formed in a frame shape having a predetermined length and positioned parallel to the ground, one end of the horizontal moving part may be coupled to the second lower bed body and the other side may be horizontally moved in the y-axis direction along the slide rail (not shown) formed in the first lower bed body 411.

Accordingly, the length of the second lower bed part 400 may be easily adjusted according to a height of the user.

FIG. 11b is a view schematically showing a second driving of the lower bed part 400 of the spinal traction device 10 according to one embodiment of the present invention when viewed from the top.

Referring to FIG. 11b, the above-described length adjusting part 422 may be fastened to at least a part of one side of the first lower bed body 411 and at least a part of the rotation guide part 412 so as to be horizontally rotatable in the widthwise direction of the lower bed part 400.

Specifically, the above-described rotation guide part 412 may be formed in a rail shape to guide the horizontal rotation of the length adjusting part 422, a sliding block (not shown) may be provided on the rotation guide part 412, and the sliding block and the length adjusting part 422 may be hinged to each other.

More specifically, although not shown in the drawings, a rhombic rotational groove (not shown) having a size corresponding to a rotational region of the length adjusting part 422 may be formed in one side of the first lower bed body 411, and the above-described slide rail may be positioned at a central part in the rotation groove.

Further, the above-described length adjusting part 422 may include two frames (not shown) overlapping each other, in which the lower frame (not shown) may have one end hinged inside the rotation groove and the other side partially hinged to the above-described sliding block so as to be rotated at a predetermined angle in parallel to the ground, the upper frame (not shown) may be movable along the lower frame so as to be horizontally moved in the y-axis direction as described above.

However, the above configuration is merely the exemplary structure for simultaneously implementing the first driving and the second driving of the lower bed part 400 and the present invention is not limited thereto. Accordingly, it is sufficient if the second lower bed member 420 has a structure horizontally rotated in the widthwise direction (x-axis direction), and simultaneously, horizontally moved in the longitudinal direction (y-axis direction).

FIG. 11c is a view schematically showing a third driving of the lower bed part 400 of the spinal traction device 10 according to one embodiment of the present invention when viewed from the front (y-axis direction in FIG. 10).

Referring to FIG. 11c, as described above, the second lower bed body may include a pair of calf support members 4211, and a horizontal slide part 4212 allowing the pair of calf support members 4211 to be horizontally moved in the widthwise direction (x-axis direction) of the lower bed part 400.

Specifically, the above-described horizontal slide part 4212 may include an upper first slide member 4212a and a lower second slide member 4212b positioned overlapping each other in the direction parallel to the ground, in which the first slide member 4212a may be horizontally moved in the widthwise direction (x-axis direction) of the lower bed part 400 on the second slide member 4212b. In addition, the above-described pair of calf support members 4211 may be coupled onto the second slide member 4212b so as to be horizontally moved together when the second slide member 4212b is horizontally moved.

As described above, the lower bed part 400 of the spinal traction device 10 according to one embodiment of the present invention is provided with two types of degrees of driving freedom in the widthwise direction, such as a horizontal rotation d2 and a horizontal movement d3, rather than a simple horizontal movement in the widthwise direction (x-axis direction), thereby expanding the rotation angle and the moving range, and the rotation angle and the moving distance are accurately adjustable by combining the two types of degrees of driving freedom, thereby increasing the lower body correction effect onto the user.

In addition, an upper surface of each of the above-described pair of calf support members 4211 may be concave downward to surround the calf of the user. Specifically, the upper surface of each of the pair of calf support members 4211 may be curved downward to correspond to an outer shape of the calf of the user. Accordingly, a comfort may be provided during seating the calf in the process of correcting the calf-spine of the user.

In addition, the above-described second lower bed member 420 may further include an ankle fixing part 423 formed at each end of the pair of calf support members 4211 to fix an ankle of the user. The above-described ankle fixing part 423 may include a pair of height adjusting shafts 4231 erected at ends of the calf support member 4211 in a direction perpendicular to the ground (z-axis direction), and a fixing member 4232 movable up and down along the pair of height adjusting shafts 4231.

The above-described fixing member 4232 descends by the user's manipulation or the automatic manipulation of the spinal traction device 10, so that an ankle part below the calf of the user seated on the calf support member 4211 may be fixed and supported.

In addition, the spinal traction device 10 according to one embodiment of the present invention may be provided with a first motor, a second motor and a third motor for the first driving, the second driving and the third driving of the above-described lower bed part 400, and each of the first to third motors may be positioned to implement each of the first driving, the second driving, and the third driving of the lower bed part 400 and driven separately and independently according to the purpose of spine correction onto the user.

In other words, the spinal traction device 10 according to one embodiment of the present invention is capable of independently operating the first driving, the second driving, and the third driving of the above-described lower bed part 400 in the above manner, so that the driving range and the degree of operational freedom of the lower bed part 400 may be improved, and the spinal correction effect onto the user may be remarkably increased.

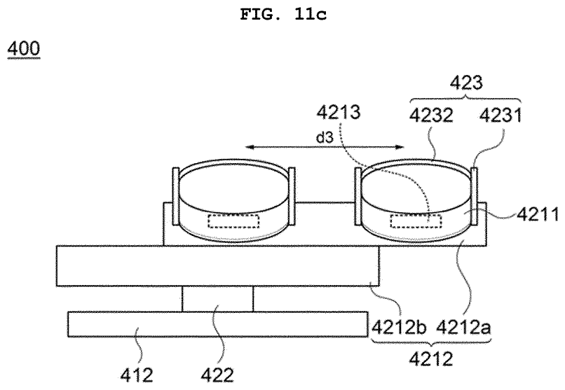

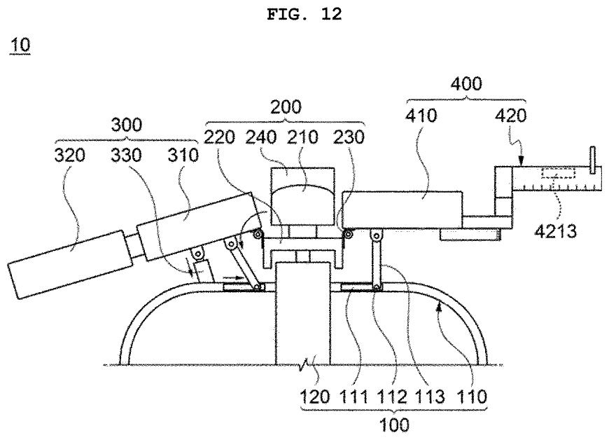

FIG. 12 is a view showing a driving of the upper bed part 300 of the spinal traction device 10 according to an embodiment of the present invention.

Referring to FIG. 12, the above-described upper bed part 300 may include a first upper bed member 310 for supporting a back of the user, a second upper bed member 320 connected to the first upper bed member 310 to support an occipital part of the user, and a driving unit 330 for rotating the first upper bed member 310 at a predetermined angle with respect to the waist bed part 200.

Specifically, the above-described driving unit 330 may be positioned such that one end of the driving unit 330 may be coupled onto the support frame 110 of the base frame 100 and the other end of the driving unit 330 may be fastened to at least a part of a lower part of the first upper bed member 310. In addition, the driving unit 330 is driven after being formed of a hydraulic cylinder, a pneumatic cylinder, or the like to have an elastic length. When the length of the driving unit 330 shrinks and shortens, the first lower bed member 410 constrained to the other end of the driving unit 330 may be lowered after being rotated at a predetermined angle around the hinge shaft of the hinge member 230.

The driving unit 330 may be operated independently of the above-described ascending and descending driving unit 120. In other words, the user may rotate only the lower bed part 400 downward through driving the driving part 330 to retract the spine based on the needs of spine traction purpose or the like, without rotating the lower bed part 300.

In addition, the above-described hydraulic or pneumatic cylinder type driving unit 330 is merely exemplary and the present invention is not limited thereto. The configuration of a cam structure, a rotary motor and a sliding scheme may be adopted, in which one end is fixed to one side of the support frame 110 and the other end is fastened to at least a part of the first upper bed member 310 and driven so as to rotate the first upper bed member 310 by a predetermined angle relative to the waist bed part 200.

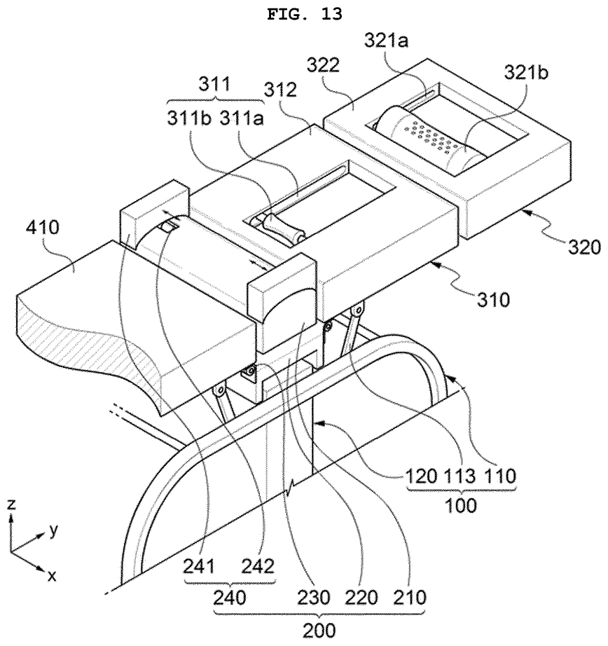

FIG. 13 is a perspective view showing the upper bed part 300 of the spinal traction device 10 according to an embodiment of the present invention.

Referring to FIG. 13, as described above, the upper bed part 300 may include a first upper bed member 310 for supporting a back part of the user, and a second upper bed member 320 for supporting an occipital part of the user. In addition, the upper bed part 300 may further include a spine scan unit 311 provided to be movable along the spine of the user on the first upper bed member 310 to detect the degree of scoliosis or lordosis of the spine.

Specifically, the above-described spine scan unit 311 may be provided at a position where the spine of the user is seated on the first upper bed body 312, and may be provided inside the first upper bed body 312 (that is, a position that does not protrude to an upper surface when viewed from the side) to prevent the user from feeling a discomfort during using the spinal traction device 10.

More specifically, the above-described spine scan unit 311 may include a pair of first guide grooves 311a formed at least a part of the inside of the first upper bed body 312, and a scan member 311b positioned between the pair of first guide grooves 311a and movable along the first guide groove 311a to detect the degree of scoliosis or lordosis of the spine of the user.

The above-described scan member 311b may be moved along a shape of the spine of the user at a position adjacent to the spine of the user through the first guide groove 311a, and preferably, may be moved in close contact with the spine of the user, so that the degree of scoliosis or lordosis of the spine of the user is detected.

In addition, the above-described scan member 311b may be configured to be three-dimensionally movable in x-axis and y-axis directions orthogonal to each other on a plane of the first upper bed member 310 and a z-axis direction orthogonal to the plane of the first upper bed member 310. In other words, the scan member 311b may be configured to be three-dimensionally movable between the pair of first guide grooves 311a, and may be movable in the x-axis, y-axis, and z-axis directions with respect to the plane of the first upper bed member 310, thereby easily moving closely along the user's spine curved in a three dimension with the scoliosis or lordosis, and detecting the degree of scoliosis or lordosis of the spine.

An ultrasonic sensing, a three-dimensional camera module or the like may be used as the scanning scheme of the scan member 311b, but this is merely illustrative and the present invention is not limited thereto.

For the three-dimensional movement described above, a pair of horizontal rails (not shown) formed along the longitudinal direction (y-axis direction) of the first upper bed member 310 may be disposed in the first upper bed body 312, and a pair of vertical rails (not shown) formed in a direction perpendicular to the ground (z-axis direction) may be disposed on the horizontal rail. The pair of vertical rails may be moved along the pair of horizontal rails in the y-axis direction, the above-described scan member 311b may be disposed on the pair of vertical rails and moved along the vertical rails in the z-axis direction.

In addition, the scan member 311b may be moved in the x-axis direction on a central axis of the scan member 311b connected to the pair of vertical rails.

However, the above-described three-dimensional movement structure of the scan member 311b is merely illustrative and the present invention is not limited thereto, and may be easily modified by those skilled in the art.

In addition, the above-described second upper bed member 320 may include a second upper bed body 322 to which the occipital part of the user may be seated, a pair of second guide grooves 321a positioned in the second upper bed body 322, and a headrest member 321b horizontally movable to a predetermined distance along the second guide groove 321a. The headrest member may be formed of an elastic material to provide convenience for the user's use, and configured to be horizontally movable along the second guide groove 321a so as to be adjusted and used according to the height of the user.

In addition, the waist bed part 200 of the spinal traction device 10 according to one embodiment of the present invention may further include a pelvic support 240. The above-described pelvic support 240 may be positioned on both sides of the waist bed member 210 in the widthwise direction (x-axis direction), and may be horizontally moved in the widthwise direction (x-axis direction) toward the pelvis of the user when the waist bed member 210 is elevated by the ascending and descending driving unit 120.

Specifically, the pelvic support 240 may include a pair of guide grooves 242 recessed to have a predetermined length in the widthwise direction (x-axis direction) of the waist bed member 210 on each side in the widthwise direction of the waist bed member 210, and a pair of pelvic support members 241 formed of an elastic material such as a cushion at an upper side of the guide groove 242. The pelvic support 240 may be provided with an additional drive motor (not shown) and operated independently of the driving of the ascending and descending driving unit 120 based on the user's selection, but the present invention is not limited thereto. When the waist bed member 210 is elevated by operating the ascending and descending driving unit 120, the pair of pelvic support members 241 may be automatically moved toward the pelvis of the user. As described above, the pelvis of the user may be fixed and supported through the pelvic support 240 during retracting the spine of the user, and the spine may be simultaneously retracted while the pelvis of the user is fixed unlike the spinal traction by simply elevating the waist bed member 210, so that the user spinal traction effect can be improved.

In addition, the width of the pelvic support member 241 in the y-axis direction has been shown to be similar to the waist bed member 210 in the current drawings, but the width is merely illustrative and the present invention is not limited thereto. The width may be about 50 mm to about 200 mm, which is wider than a width of the waist bed member 210 in the y-axis direction. Accordingly, when the waist bed 200 is elevated, the pelvis of the user may be easily fixed and supported in a wide region.

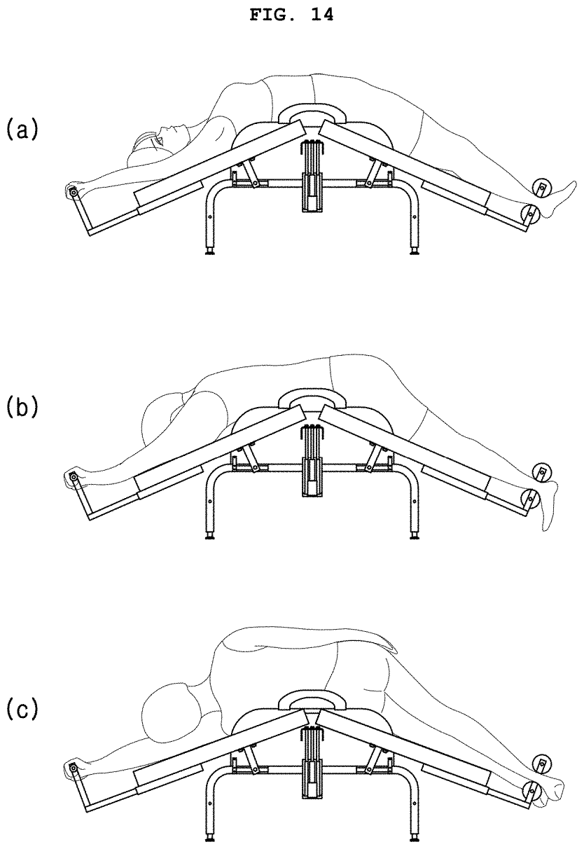

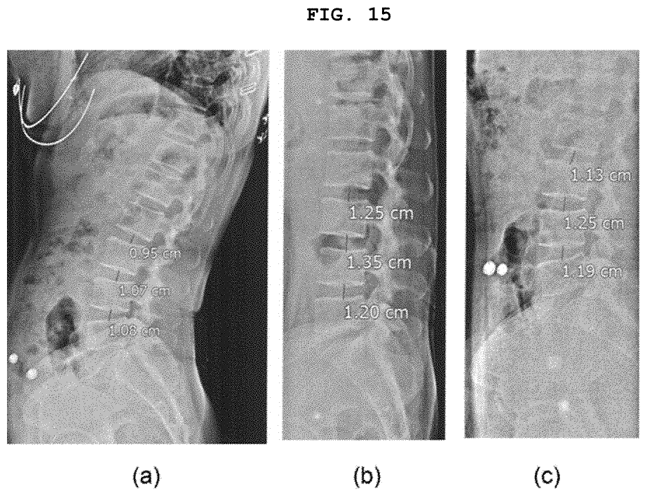

FIGS. 14(a) to 14(c) are photographs showing examples of a used state of the spinal traction device 10 according to one embodiment of the present invention. FIG. 15(a) is an x-ray photograph showing a state of a spine before using the spinal traction device 10 according to one embodiment of the present invention. FIG. 15(b) is an x-ray photograph showing a state of the spine when a user uses the spinal traction device 10 in a supine posture according to one embodiment of the present invention. FIG. 15(c) is an x-ray photograph showing a state of the spine when the user uses the spinal traction device 10 in a prone posture according to one embodiment of the present invention.

Specifically, FIG. 14(a) is a photograph showing an example of a used state of the spinal traction device 10 according to one embodiment of the present invention while the user is in a supine posture (extension exercise). FIG. 14(b) is a photograph showing an example of a used state of the spinal traction device 10 according to one embodiment of the present invention while the user is in a prone posture (flexion exercise). FIG. 14(c) is a photograph showing an example of a used state of the spinal traction device 10 according to one embodiment of the present invention while the user is in a flank posture (flank exercise).

Referring to FIGS. 14 and 15, the user may use the spinal traction device 10 according to one embodiment of the present invention in various ways. Actually, it was found that the spinal traction is effectively performed when the spinal traction device 10 according to one embodiment of the invention is used. Table 1 shows the comparison results of spinal states before and after the spinal traction device 10 according to one embodiment of the invention is used as follows.

TABLE 1

| Spine | | | | |
|---|---|---|---|---|
| (lumbar | | | After supine posture | After prone posture |
| vertebra) | Before exercise | | (extension exercise) | (flexion exercise) |
| L2 to L3 | 9.5 | mm | 11.3 mm | 12.5 mm |
| | | | (1.8 mm extension) | (3 mm extension) |
| L3 to L4 | 10.7 | mm | 12.4 mm | 13.5 mm |
| | | | (1.8 mm extension) | (2.8 mm extension) |
| L4 to L5 | 10.8 | mm | 11.9 mm | 12 mm |
| | | | (1.1 mm extension) | (1.2 mm extension) |

As described above, the spinal traction device 10 according to one embodiment of the invention may align the spine correctly through a natural traction effect in a curved state, so that various spinal diseases can be prevented, and the growth of a child during a growth period can be facilitated.

In addition, the spinal traction device 10 according to one embodiment of the invention can be used for various types of exercises, such as a supine posture, a prone posture, and a flanked posture, and effects such as prevention and treatment of visceral diseases (the supine posture), removal of abdominal obesity (the prone posture), and stimulation of digestion, respiration and excretion (the flank posture) can be obtained through each exercise posture.

Although the present invention has been described in detail with reference to the exemplary embodiments, it will be apparent that a person having ordinary skill in the art may carry out various deformations and modifications for the embodiments described as above within the scope without departing from the present invention. Therefore, the scope of the present invention should not be limited to the aforementioned embodiments, and should be determined by all deformations or modifications derived from the following claims and the equivalent thereof.

### INDUSTRIAL APPLICABILITY

The present invention may be applicable in the field of a spinal traction device.

1. A spinal traction device comprising:
a base frame;
a waist bed part provided on an upper side of the base frame so as to be movable up and down with respect to a ground to support a waist of a user;
an upper bed part connected to one side of the waist bed part to support an upper body of the user; and
a lower bed part connected to an opposite side of the waist bed part to support a lower body of the user, the lower bed part comprising a first lower bed member for supporting a thigh of the user and a second lower bed member for supporting a calf portion of the user, the second lower bed member horizontally movable in a longitudinal direction of the lower bed part with respect to the first lower bed member and horizontally movable or horizontally rotatable in a widthwise direction perpendicular to the longitudinal direction,
wherein the upper bed part and the lower bed part are rotated at a predetermined angle with respect to the waist bed part when the waist bed part moves up and down with respect to the ground.

2. The spinal traction device of claim 1, wherein the second lower bed member comprises:
a second lower bed body; and
a length adjusting part connecting the first lower bed member to the second lower bed body, the length adjusting part being horizontally movable in the longitudinal direction of the lower bed part to adjust a length of the lower bed part.

3. The spinal traction device of claim 2, wherein the first lower bed member comprises:
a first lower bed body; and
a rotation guide part provided on the first lower bed body to guide a horizontal rotation of the second lower bed member,
wherein the length adjusting part is rotatably fastened to the first lower bed body and at least a part of the rotation guide part along the rotation guide part so as to be horizontally rotatable in the widthwise direction of the lower bed part.

4. The spinal traction device of claim 2, wherein the second lower bed body comprises:
a pair of calf support members spaced apart from each other to support calves of the user, respectively; and
a horizontal slide part capable of horizontally moving the pair of calf support members in the widthwise direction of the lower bed part from one side of the length adjusting part.

5. The spinal traction device of claim 4, wherein the second lower bed body further comprises:
sensors positioned at the pair of calf support members to detect distances between the pair of calf support members and between the calves of the user, respectively.

6. The spinal traction device of claim 4, wherein an upper surface of each of the pair of calf support members is concave downward to surround the calf of the user.

7. The spinal traction device of claim 4, wherein horizontal positions of the pair of calf support members are higher than a horizontal position of the first lower bed member with respect to the ground.

8. The spinal traction device of claim 1, wherein the upper bed part comprises:
a first upper bed member for supporting a back of the user;
a second upper bed member connected to the first upper bed member to support an occipital part of the user; and
a driving unit for rotating the first upper bed member by a predetermined angle with respect to the waist bed part.

9. The spinal traction device of claim 1, wherein the upper bed part further comprises:
a spine scanner provided to be movable along the spine of the user on the first upper bed member to detect a degree of scoliosis or lordosis of the spine.

10. The spinal traction device of claim 9, wherein the spine scanner comprises:
a guide groove; and
a scan member movable along the spine of the user through the guide groove.

11. The spinal traction device of claim 10, wherein the scan member is three-dimensionally movable in x-axis and y-axis directions orthogonal to each other on a plane of the first upper bed member and a z-axis direction orthogonal to the plane of the first upper bed member.

12. The spinal traction device of claim 1, wherein the waist bed part comprises:
a waist bed body; and
a pelvic support provided on the waist bed body so as to be moved toward a pelvis of the user when the waist bed part ascends.

13. A spinal traction device comprising:
a pair of supports having guide rails in front and rear directions of an inner surface thereof;
a lower bracket with a cylinder and a guide rod that are fixed between the pair of supports and vertically operated;
an upper bracket on the lower bracket for fixing the cylinder and the guide rod;
an upper bed and a lower bed provided with hinges in front and rear directions of the upper bracket and rotatable about the hinges, respectively; and
connecting rods having rollers attached to lower ends on both sides of middle parts of the upper bed and the lower bed in which the rollers move along the guide rails, respectively.

14. The spinal traction device of claim 13, further comprising:
a spine support provided between the upper bed and the lower bed; and
cushions provided on both sides of the spine support.

15. The spinal traction device of claim 13, wherein the upper bed has a space, and a headrest is provided in the space and is movable along guide grooves on both sides of the space part.
